(19) United States
(12) Patent Application Publication
(10) Pub. No.: US 2004/0050701 A1
(43) Pub. Date: Mar. 18, 2004

(54) Electrostatically guiding ionized droplets in chemical array fabrication

(76) Inventors: John Francis McEntee, Boulder Creek, CA (US); Jay Kevin Bass, Mountain View, CA (US); Alexander Scott Williamson, Palo Alto, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/243,505

(22) Filed: Sep. 13, 2002

(51) Int. Cl. C12M 1/34

(52) U.S. Cl. 204/465

(57) ABSTRACT

An apparatus, method and system electrostatically guide ionized droplets of a material in chemical or biological array fabrication. The apparatus comprises a plurality of deposition sites and an area surrounding the plurality of sites on a surface of a substrate. An electrostatic charge differential is generated on the apparatus between a selected set of deposition sites and the surrounding area remaining around the selected set. In some embodiments, the substrate comprises a photoconductive layer overlying a conductive layer. In other embodiments, the substrate comprises circuit paths interconnecting to electrode pads defined in the substrate. The electrode pads correspond to the plurality of deposition sites. The substrate may further comprise a membrane sheet that supports the deposition sites. The method further comprises exposing the array to the ionized droplets after the electrostatic charge differential is generated. The apparatus provides an electrostatically changeable deposition mask. The system comprises the apparatus.

### TECHNICAL FIELD

This invention relates to chemical arrays. In particular, the invention relates to chemical array manufacture using electrostatically guided droplets.

### BACKGROUND ART

Droplet generators are used to deposit small amounts of different materials to deposition surfaces. The droplet generator can be a spraying apparatus, a fogger, or a precision applicator, such as an ink-jet print head.

An ink-jet print head is an element of an ink-jet printing device, which has the ability to project droplets of liquid material through nozzles and onto a surface, such that the generated droplets are launched in a trajectory to the surface. These devices are commonly used for printing ink onto paper, but are also used to dispense many chemical and biological materials, especially in chemical array manufacture. There are two common types of ink-jet heads, differing primarily in the mechanism used to produce the mechanical energy necessary to project the droplets. The first type of print head is sometimes called bubble-jet print head. It relies on a propellant in the liquid or ink that can be vaporized by a small electric resistive heater. When circuitry at the nozzle is activated, the microscopic heater creates a small vapor bubble, which displaces the liquid, thereby forcing the liquid through the nozzle where it is accelerated and projected as a small droplet onto the deposition surface. The bubble in the ink-jet print head rapidly collapses when the vapor suddenly cools and condenses. The second type of ink-jet print head uses a piezoelectric device to displace the liquid. A piezoelectric material in the nozzle changes shape when an electric field is applied thereto. When the electric field is applied, the piezoelectric material deforms to push the liquid in the form of a droplet out of the nozzle. As mentioned above, the generated droplets are launched or projected in a trajectory to a surface. The trajectory of a generated droplet controls the final position of the droplet on the surface. For conventional ink-jet equipment, the final position of a droplet on a surface is controlled only by the trajectory that the droplet takes from the ink-jet nozzle to the surface. Unfortunately, due to the manufacturing variations of the ink-jet nozzle and the buildup of deposits on the nozzle over time, variations in the final droplet positions are inevitable.

A spraying apparatus forces a liquid under pressure through a fine nozzle or an array of nozzles. The forced liquid generates liquid droplets from the nozzle. The droplets may move to their target in an unaided ballistic path, or they may be entrained in an airflow. The most common spraying apparatus is of the aspirator type. An aspirator apparatus produces small droplets by guiding an air stream across an orifice of a liquid inlet, thereby producing a vacuum and thus, drawing the liquid into the air stream. The liquid is broken into a spray of small droplets by the air stream. Another type of spraying apparatus accelerates a liquid stream through a small orifice with short bursts of pressure. These and other spraying apparatuses are well known in the art.

A fogger or aerosol generator is used to generate small liquid droplets (i.e., microdroplets) in a cloud. Fogs can be created by heating a liquid to the vapor phase and then cooling the vapor until condensation occurs. Other methods of generating a fog include atomizing the liquid into small droplets by launching high frequency sound into the liquid, such as by a piezoelectric device. These and other aerosol generators also are well known in the art.

Each of these droplet generators may be used to produce chemical arrays. Chemical arrays are comprised of a plurality of probes applied in a regular or spatially addressable pattern on a substrate. The probes are small spots or features of reagent, genetic, chemical or biological materials, which typically are used to test chemical or biological reactions. The precision with which the features correspond to a desired pattern can be important to the successful function of the array in an assay or test. Smaller features and closer packing of the features can allow a single array to contain more tests in a given area. This is desirable because smaller arrays utilize fewer materials in their manufacture and can react to smaller test samples sizes in their use. However, smaller features and closer packing of the features require higher precision to produce a spatially addressable pattern.

The droplets projected from an ink-jet nozzle are launched with the intent that they arrive at a predetermined deposition position. However, the precision of the ink-jet technology is based on how much a droplet actually overlaps the predetermined position. The same is true for the other droplet generation equipment. The distance between the intended deposition position and the final droplet position defines the placement error. Placement errors using ink-jet technology can be the result of a number of factors, such as vibration, variations in nozzle precision, air turbulence in the gap between the printhead and the deposition surface, and residues built up at the nozzle exit. Placement errors are a source of errors in final assay results. Assay errors are quite costly, since the expensive materials, scarce test samples, time and labor have already been expended. Further, placement errors significantly impact the ability to make the desirable smaller arrays that have more closely packed probe features.

Thus, it would be advantageous to have a technique that reduces the uncertainty in the placement of droplets using conventional droplet generator equipment. Such a technique would solve a long-standing problem in the art of making chemical arrays.

### SUMMARY OF THE INVENTION

The present invention electrostatically guides a droplet of a material to a location on an array of deposition sites. The present invention addresses droplet placement errors in the art when conventional equipment is used to generate droplets for chemical array fabrication. The droplet is electrostatically guided to a specific location on the array using both droplet ionization and a charge-influenced deposition site on the array.

An apparatus that electrostatically guides a chemical or biochemical material to be deposited in chemical array fabrication is provided. The apparatus comprises a substrate that comprises a photoconductive layer overlying an electrically conductive layer, and an electric charge differential generated on the surface of the photoconductive layer by selectively illuminating the surface with light. The electric charge differential induces or guides a droplet of the chemical or biochemical material having a charge to preferentially deposit on a location of the surface due to an electrostatic force.

Another apparatus that electrostatically guides a chemical or biochemical material to be deposited in chemical array fabrication is provided. The apparatus comprises a substrate that comprises a plurality of deposition sites spatially arranged in an array pattern on an array surface, and circuitry defined in the substrate that electrically influences the plurality of deposition sites. When a charge is supplied to the circuitry, a deposition site is influenced by the charge and an electric charge or field differential is created at the array surface. The electric charge or field differential guides an ionized droplet of the material to preferentially deposit on the electrically influenced deposition site or another location on the array surface due to an electrostatic force.

A deposition mask having an electrostatically changeable mask pattern is provided. In an embodiment, the deposition mask comprises the apparatus having a substrate including a photoconductive layer overlying a conductive layer. In this embodiment, the changeable mask pattern comprises a pattern of different electric charges for each selectively illuminated set of locations, a selected set of illuminated locations being provided for each chemical or biochemical material to be deposited and for each incidence that a same chemical or biochemical material is deposited during the chemical array fabrication. Each selected set has at least one illuminated location, wherein the at least one illuminated location is a same location or a different location relative to other selected sets. In another embodiment, the deposition mask comprises the apparatus having a substrate including a plurality of deposition sites and circuitry that electrically influences the plurality of sites. Each selected set has at least one deposition site, wherein the at least one deposition site of each selected set is one or both of a same deposition site and a different deposition site of the plurality relative to other selected sets.

A system for electrostatically guiding a chemical or biochemical material in chemical array fabrication is provided. In an embodiment, the system comprises the apparatus having a substrate including a photoconductive layer overlying a conductive layer. In this embodiment, the system further comprises one or more of a light source that provides the selective illumination to the surface; a source of charge or a sink for charge that provides an electric charge of the differential; and a droplet generator that provides the charged droplet of the chemical or biochemical material to the apparatus. In another embodiment, the system comprises the apparatus having a substrate including a plurality of deposition sites and circuitry that electrically influences the deposition sites. In this embodiment, the system further comprises a source of ionized droplets of the material; and control electronics comprising the source of charge. The control electronics one or more of supplies, removes and varies the charge applied to the circuitry. The control electronics optionally comprises charge-sensing circuitry that monitors a volume of the material that is accreted on the substrate surface.

A method of electrostatically guiding a chemical or biochemical material to be deposited in chemical array fabrication is provided. The method comprises generating an electric charge or field differential on an array; and exposing the array to an ionized droplet of the chemical or biochemical material. The array comprises a plurality of deposition sites on a surface of the array. The plurality of deposition sites is in a spatially addressable array pattern. The electric charge or field differential comprises a first charge that electrically influences a selected set of deposition sites of the plurality, and a second charge on an area surrounding the selected set. The first charge is different from the second charge. The ionized droplet has a charge, such that the electric charge or field differential guides the material to preferentially deposit on either the selected set of influenced deposition sites or the surrounding area due to an electrostatic force.

One or more of the following advantages may be realized by using the present invention. Higher precision deposition and volume control of droplets are possible from conventional ink-jet print heads, aerosol generators and sprayers than that provided without the present invention. The charged, or otherwise charged-influenced, surface patterns on the substrate essentially force the droplets into predefined regions, making the final position of the material more precise. When deposition is more precise, higher density array patterns that are denser than that conventionally used can be designed. When a higher density array pattern is used, the deposited materials, such as expensive biological probe materials, can be made smaller and more closely spaced, since placement error is reduced. Since the droplets can be smaller, less material is used for each chemical array, and less material is wasted. Moreover, the ‘soft’ deposition mask created by the changeable mask pattern of electric charges can be used for the mass deposition of materials according to the present invention. Separate physical masking steps using traditional masking materials are not necessary. After a deposition process, the charge applied to the pattern mask of the present invention can be removed and reapplied without the application of physical materials or requiring a chemical process. Certain embodiments of the present invention have other advantages in addition to and in lieu of the advantages described hereinabove. These and other features and advantages of the invention are detailed below with reference to the following drawings.

### BRIEF DESCRIPTION OF THE DRAWINGS

The various features and advantages of the present invention may be more readily understood with reference to the following detailed description taken in conjunction with the accompanying drawings, where like reference numerals designate like structural elements, and in which:

FIG. 1 illustrates a cross sectional side view of an apparatus that electrostatically guides a material in chemical array fabrication according to an embodiment of the present invention.

FIG. 2A illustrates a cross sectional side view of the apparatus embodiment of FIG. 1 when illuminated with light.

FIG. 2B illustrates a cross sectional view of the apparatus embodiment of FIG. 1 after the illumination in FIG. 2A.

FIG. 2C illustrates a perspective view of the apparatus embodiment of FIGS. 2A and 2B.

FIG. 3 illustrates a perspective view of the apparatus embodiment of FIG. 2B after illumination is complete.

FIG. 4A illustrates a cross sectional side view of a portion of the apparatus embodiment of FIG. 3 with an ionized droplet deposited on an illuminated region.

FIG. 4B illustrates a perspective view of ionized droplets placed on the illuminated regions of the apparatus embodiment of FIG. 3.

FIG. 5A illustrates a perspective view of an apparatus that electrostatically guides a material in chemical array fabrication according to another embodiment of the present invention.

FIG. 5B illustrates a perspective view of an apparatus that electrostatically guides a material in chemical array fabrication according to another embodiment of the present invention.

FIG. 6 illustrates a cross sectional side view of an apparatus that electrostatically guides a material in chemical array fabrication according to another embodiment of the present invention.

FIG. 7 illustrates a block diagram of a method of electrostatically guiding a material in chemical array fabrication according to an embodiment of the present invention.

FIG. 8 illustrates a block diagram of a system for electrostatically guiding a material in chemical array fabrication according some embodiments of the present invention.

FIG. 9 illustrates a block diagram of an embodiment of the system of FIG. 8 including the apparatus embodiment of FIG. 1.

FIG. 10A illustrates a perspective view of an embodiment of the system illustrated FIG. 8 including the apparatus embodiment of either FIGS. 5A or 5B.

FIG. 10B illustrates a cross sectional side view of an embodiment of the system including the apparatus embodiment of FIG. 6.

FIG. 10C illustrates a magnified view of a section labeled 10C of the embodiment of the system illustrated in FIG. 10B.

### DETAILED DESCRIPTION

### Definitions

In the present application, unless a contrary intention appears, the following terms refer to the indicated characteristics. A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and the references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A biomonomer fluid or a biopolymer fluid reference a liquid containing either a biomonomer or biopolymer, respectively (typically in solution). A “biochemical” refers to a biomonomer, a biomonomer fluid, an oligonucleotide, an oligonucleotide fluid, a biopolymer, a biopolymer fluid, or any reagent used in the fabrication of a biological array. A “chemical” refers to any and all chemical substances used in the fabrication of a chemical array, including biochemicals used in the fabrication of a biological array.

An “array”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one that is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. “Hybridizing” and “binding”, with respect to polynucleotides, are used interchangeably. A “region” refers to any finite small area on the array that can be illuminated and any resulting fluorescence therefrom simultaneously (or shortly thereafter) detected, for example a pixel.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

Reference to a singular item, includes the possibility that there are plural of the same items present. “May” means optionally. Methods recited herein may be carried out in any order of the recited events, which is logically possible, as well as the recited order of events. All patents and other references cited in this application are incorporated into this application by reference except insofar as they may conflict with those of the present application (in which case the present application prevails).

### Array Description

Any given substrate may carry one, two, three, four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments, each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features may be of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, the references including U.S. Pat. Nos. 6,242,266; 6,232,072; 6,180,351; 6,171,797; and 6,323,043; U.S. patent application Ser. No. 09/302,898, filed Apr. 30, 1999, by Caren et al.; and the references cited therein. As already mentioned, these references are incorporated herein by reference. Other drop deposition methods can be used for fabrication, as previously described herein. Also, instead of drop deposition methods, photolithographic array fabrication methods may be used, such as described in U.S. Pat. Nos. 5,599,695; 5,753,788; and 6,329,143. Interfeature areas need not be present particularly when the arrays are made by photolithographic methods as described in those patents.

### Reading Array Material

Following receipt by a user, an array made by an apparatus or a method of the present invention will typically be exposed to a sample (for example, a fluorescently labeled polynucleotide or protein containing sample) and the array then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications Ser. No. 10/087,447, “Reading Dry Chemical Arrays Through The Substrate” by Corson et al.; and Ser. No. 09/846,125, “Reading Multi-Featured Arrays” by Dorsel et al. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685 and 6,221,583, and elsewhere). A result obtained from the reading may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

### Modes For Carrying Out The Invention

The present invention provides for electrostatically guiding droplet placement onto an array surface in chemical or biological array fabrication. The present invention creates differences in electric charge and/or electric field at the array surface on which the droplets are deposited, so that the droplets favor desired positions based on the charge or the electric field. The droplets can comprise one or more of a liquid medium and an aerosol medium, for example. The droplets are ionized, such that they have an overall charge polarity. The composition of the droplets is dependent on the intended use of the droplets and the array and is not a limitation to the invention. For example, the droplets can be any one or more of a variety of chemicals and/or biochemicals that are used for a variety of purposes. In particular, the material of the droplets may be a reagent, a chemical composition, and/or a biochemical composition used in the fabrication or manufacture of chemical or biological probes arrays. Further, the material droplet may be used in the subsequent processing of the chemical or biological probe arrays for at least one or more of analytical, diagnostic, and therapeutic purposes.

Accordingly, an apparatus that electrostatically guides ionized droplets of a material to be deposited in chemical array fabrication is provided. In some embodiments, the apparatus comprises a substrate that comprises a photoconductive layer overlying a conductive layer. The conductive layer is electrically conductive, while the photoconductive layer is an electrical insulator or is electrically nonconductive in the absence of light.

The insulative or nonconductive photoconductive layer is chargeable with an electrostatic charge on an array surface of the photoconductive layer. The array surface is an exposed surface of the photoconductive layer, which is opposite to a surface of the photoconductive layer that is adjacent to the underlying conductive layer. Moreover, the photoconductive layer can be made electrically conductive in a location by illuminating the location with light, thereby allowing the electrostatic charge on the array surface to pass or dissipate through the photoconductive layer to the conductive layer at the illuminated location. The charge on the illuminated location is therefore different from the electrostatic surface charge on a remaining non-illuminated surface area. An electric charge differential and pattern thereof are created on the array surface when a location is illuminated with light. The electric charge differential and pattern thereof can be effectively erased and reapplied as the same or a different pattern by discharging the charged surface, reapplying an electrostatic surface charge on the array surface of the photoconductive layer and illuminating the same or a different location on the photoconductive layer with light. Any photoconductor material may be useful for these embodiments of the present invention including, but not limited to, a calcognide class of materials; organic photoconductors (OPC); amorphous silicon; selenium alloys, such as selenium tellurium (SeTe); zinc oxide; calcium sulfide; and pure sulfur, for example, provided that the particular photoconductive material used does not interfere with the chemistry of chemical or biological array fabrication, including in situ oligonucleotide synthesis and other microarray chemistry. Other photoconductive materials not listed herein, which are known to those skilled in the art, also may be within the scope of the present invention.

For the purposes of the present invention, a photoconductive material differs from other photosensitive materials in that other photosensitive materials use the photoelectric effect to charge a surface of the material, as described by Loewy et al. in U.S. Pat. No. 6,004,752. In contrast, a photoconductive material uses a photoconductive effect, wherein photoconductive material resists the passage of electric current unless exposed to light. In the photoelectric effect, free electrons are stripped or emitted from a surface of the photosensitive material when subjected to light, giving a positive charge to the surface of the photosensitive material. However in the photoconductive effect, the light changes the electrical conductivity of the photoconductive material from being electrically insulating to being electrically conductive in the illuminated location. When the conductivity is changed during illumination, a charged surface of the photoconductive layer is discharged to a conductive layer through the electrically conductive illuminated location of the photoconductive layer. The photoconductive effect is used in Xerography, for example. This electrically conductive feature of the photoconductive material allows the surface at the illuminated location to acquire the charge present on the conductive layer.

An array pattern of locations can be defined on the surface of the photoconductive layer with light. The array pattern of locations comprises a plurality of deposition sites on the array surface of the photoconductive layer. The plurality of deposition sites is spatially arranged in an array pattern that is spatially addressable on the array surface. Either a deposition site or an area surrounding the deposition site corresponds to or is an illuminated location, whether the location is a presently illuminated location, a previously illuminated location or a subsequently illuminated location (i.e., ‘subsequently’ as in ‘to be’ illuminated for fabrication of a chemical array). The light illuminates the deposition site or the surrounding area until the electrical conductivity of the illuminated location changes and an electric charge changes on the array surface of the photoconductive layer at the illuminated location. The period of time for the illumination to effect a change in conductivity and in electric charge will depend on the photoconductive material chosen and is not intended to limit the scope of the present invention. The period of time may be referred to herein as ‘momentary’ with the understanding that this ‘momentary’ period of time is preferably. The conductivity of the material at the location returns to being electrically nonconductive when the illumination is removed. The different charge on the array surface at the illuminated location will eventually discharge but will remain for a limited time, depending on the photoconductive material. The ‘limited time’ is preferably at least an amount of time sufficient to electrostatically guide ionized droplets in accordance with the invention. Either a deposition site or a surrounding area is distinguished or delineated for each electric charge differential pattern to be created. For the purposes of simplicity only, and not by limitation, hereinafter the illuminated location is generally referred to as the deposition site or the plurality of deposition sites. While the deposition site or the plurality of deposition sites is referred to as being the illuminated location(s) to change the electric charge, it should be understood that either a deposition site or the area surrounding the deposition site can be illuminated to achieve the electric charge differential or a charge differential pattern according to the invention.

Each deposition site is rendered electrically conductive and is effectively electrically connected to the conductive layer when illuminated with light, such that the illuminated deposition site can acquire the electric charge of the underlying conductive layer. The conductive layer is chargeable with a first electric charge that electrically influences the illuminated or distinguished deposition sites of the plurality. The conductive layer may be attached to ground, such that first electric charge is of zero potential or a neutral charge. Alternatively, the conductive layer may have a charge with a positive polarity or a negative polarity and a potential greater than zero applied thereto. The electric charge on the conductive layer is preferably an electrostatic charge, although a non-static or variable electric charge on the conductive layer is within the scope of the present invention. The insulative surface of the photoconductive layer is chargeable with a second electric or electrostatic charge, which is different from the first charge. For example, the insulative surface may have no charge provided thereto, such that the second charge is of zero potential or a neutral charge. Alternatively, the insulative surface may have a charge with a potential greater than zero and a positive or a negative polarity. The electric charge on the insulative surface is an electrostatic charge. The difference between the first charge and the second charge may be a difference in charge polarity and/or a difference in charge potential. Preferably, the charge polarities are different.

During chemical or biological array fabrication, the chemical or biochemical material is deposited as an ionized droplet that also has a charge. The charge on the ionized droplets facilitates electrostatic guidance according to the invention. When the electric charge differential is generated or created on the surface of the photoconductive layer by selective illumination, as described above, the electric charge differential guides the ionized droplet of the material to preferentially deposit on an illuminated location or a non-illuminated location of the surface, such as either a deposition site or the non-illuminated remainder of the photoconductive layer surface, due to an electrostatic force. Consequently, the deposited material chemically bonds to the photoconductive layer surface at the preferred location.

In other embodiments, an apparatus comprises an electrically insulative substrate comprising a spatially arranged and/or spatially addressable array pattern of feature locations or deposition sites on an array surface of the apparatus. An area of the array surface that essentially surrounds the spatially arranged deposition sites is relatively nonconductive. The substrate further comprises electrically conductive circuitry associated with the plurality of deposition sites. The circuitry is defined in the substrate. By ‘defined in the substrate’, it is meant that the circuitry, or a portion thereof, is embedded in the substrate or provided adjacent to a surface of the substrate. By ‘adjacent’ it is meant that the circuitry, or a portion thereof is on or just under the surface. The circuitry is electrically connectable to a source of charge and is electrically chargeable. Generally, the source of charge is external to the substrate. The circuitry is ‘associated with’ the plurality of deposition sites in that the circuitry either directly or indirectly electrically influences a deposition site of the plurality with an electric charge from the source of charge. By ‘electrically influence’ it is meant that a deposition site acquires the electric charge of the circuitry or the electric charge applied to the circuitry generates an electric field at a deposition site.

Hereinafter, the electric charge provided to the circuitry to directly or indirectly influence a deposition site is a first electric charge. The surrounding area of the array surface may or may not be charged with an electrostatic surface charge. Hereinafter, the electrostatic surface charge on the surrounding area is called a second charge, which is different from the first charge. The second charge may be of zero potential (or have a neutral charge), such as when the surrounding surface is not charged, or have a positive or negative polarity and a potential greater than zero, when the surrounding surface is charged. Moreover, the first charge may be zero potential (or have a neutral charge), such as when the circuitry is attached to ground, or have a positive or negative polarity and a potential greater than zero. Preferably, the first charge is a variable or non-static charge as compared to an electrostatic charge, but an electrostatic first charge is within the scope of these embodiments. The first charge differs from the second charge in the charge polarity and/or the charge potential. Preferably, the first charge has different charge polarity from the second charge.

During chemical array fabrication using these embodiments of the apparatus, a chemical or biochemical material is deposited on the array surface as an ionized droplet of the material having a charge. The charge on the ionized droplet facilitates electrostatic guidance according to the invention. When one or both of the first charge and the second charge is applied to the substrate, an electric charge or field differential is created on the array surface. The electric charge or field differential guides the ionized droplet of the material to preferentially deposit on either a deposition site or the surrounding area due to an electrostatic force. Consequently, the deposited material chemically bonds to the array surface at the preferred location.

In either apparatus embodiments of the present invention, the first charge and the second charge preferably are of opposite polarity to each other. In some embodiments of the apparatus, either the first charge or the second charge is zero or neutral, when the other of the first charge and the second charge is not. The ionized droplets of the material to be deposited are generated from any one or more of the conventional droplet generators discussed herein, but the droplets are also ionized before, during or after droplet generation. The charge of the ionized droplets facilitates electrostatic guidance of the ionized droplet to a desired location on the apparatus. For example, the charge of the ionized droplet is made sufficiently different from the first charge or the electric field created by the first charge, such that the ionized droplet is electrostatically attracted to a deposition site influenced by the first charge rather than a surrounding area. Moreover or alternatively, the charge of the ionized droplets is made sufficiently similar to the second charge, such that the ionized droplet is electrostatically repelled by the surrounding area into a deposition site. As such, the electrostatic force guiding the ionized droplet to a particular location on the array surface is an attractive force, a repulsive force, or both an attractive force and a repulsive force.

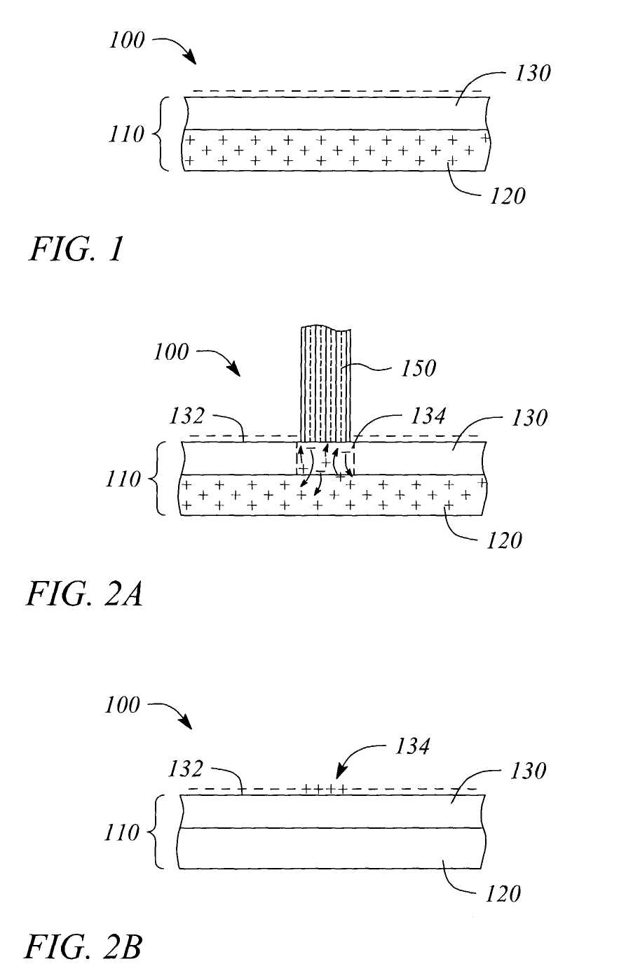

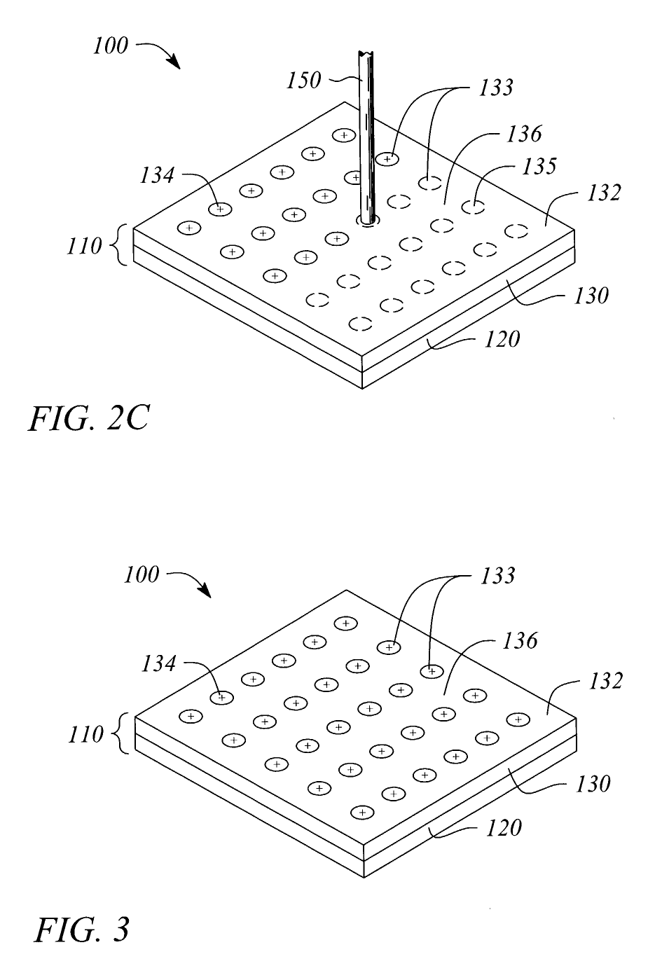

FIG. 1 illustrates a cross sectional view of an apparatus 100 according to some embodiments. The apparatus 100 comprises a substrate 110 that comprises a photoconductive layer 130 overlying a conductive layer 120. The substrate 110 may further comprise additional layers (not shown) for chemical, biological, mechanical, structural or other purposes. As mentioned above, the conductive layer 120 is electrically conductive, and the photoconductive layer 130 is essentially electrically nonconductive or an insulator in the absence of light. In a location on the photoconductive layer 130 that is illuminated with light, the photoconductive layer 130 becomes momentarily electrically conductive, thereby allowing electric charge to pass through the photoconductive layer 130 at the illuminated location during the illumination, as illustrated in FIG. 2A. While momentarily illuminated, the electrically conductive location is effectively connected to the conductive layer 120 to change the charge of the illuminated location. The conductive layer 120 is selectively connectable to ground and/or a source of charge. The illuminated location acquires the charge of the conductive layer during illumination.

In some embodiments, the conductive layer 120 is a solid, continuous conductive plate and the photoconductive layer 130 is an insulative layer on the conductive plate 120. In some of these embodiments, once a chemical array of biopolymers is fabricated on the photoconductive layer 130 surface, the photoconductive layer 130 is separable from the conductive plate 120 for use in an subsequent assay, for example a hybridization assay. In others of these embodiments, the conductive layer 120 is a solid, continuous layer that remains with the photoconductive layer 130. Once the chemical array of biopolymers is fabricated on the photoconductive layer surface, the conductive layer 120 provides support to the photoconductive layer 130 during a subsequent assay.

However, it is within the scope of this embodiment of the apparatus 100 for the conductive layer 120 to be a circuit layer that comprises a pattern of circuitry on an electrically insulative support. The circuitry pattern comprises conductive circuit pads at least underlying each of the illuminated locations and conductive circuit paths that ultimately and selectively connect the circuit pads to ground and/or a source of charge (not shown). The conductive layer 120 (in particular, the circuit layer embodiment) provides for selective application of differing first charges to selected sites of the illuminated deposition sites. Therefore for the purposes of the present invention, reference to the ‘conductive layer 120’ includes the conductive plate and the circuit layer described above and is not intended to limit the scope of the present invention to only the solid, continuous conductive layer embodiments.

As mentioned above, the conductive layer 120 is chargeable with a first charge. The insulative photoconductive layer 130 is chargeable on the surface with a second charge that is different from, and preferably has an opposite polarity to the first charge. Also as mentioned above, the photoconductive effect permits a statically charged surface on the non-illuminated photoconductive layer 130 to be discharged or dissipated to the conductive layer 120 through the photoconductive layer 130 at an illuminated location 134 during illumination. The conductive illuminated location 134 acquires the charge of the underlying conductive layer 120, as illustrated in FIG. 2A.

For example, consider that the photoconductive layer 130 has second charge of a negative polarity applied to a surface 132 thereof, while the underlying conductive layer 120 has a first charge of a positive polarity applied thereto. Depending on the application, the charge polarities on the conductive layer 120 and the photoconductive layer 130 could be reversed, or the conductive layer 120 could be connected to ground, without altering the concept of the present invention. FIGS. 1 and 2A illustrate a positive first charge applied to the conductive layer 120 and a negative second charge applied to the surface 132 for the purpose of this example only. The first charge can be applied to the conductive layer 120 through a direct electrical connection to a voltage source, a current source or other charge generating means or source of charge known in the art. The voltage or current source preferably has a zero volts or zero amps selection to optionally and effectively connect the conductive layer 120 to ground, when desired. Alternatively, the first charge may be applied to the conductive layer 120 via any indirect means for generating a charge known in the art, without limiting the scope of the present invention.

To make the apparatus 100, the photoconductive layer 130 is protected from ambient light during the charging processes. A negative or positive second charge is applied to the surface 132 of the photoconductor layer 130 in the absence of light (i.e., while in the insulating state), for example using a corona wire or other technique known to one skilled in the art. For applying an electrostatic charge to a material, see for example, the techniques described in U.S. Pat. No. 6,004,752, incorporated by reference in its entirety herein.

Moreover, the conductive layer 120 is electrically connected to ground or a source of charge to produce a first charge that is different from the second charge in the conductive layer 120 while the photoconductive layer 130 is protected from ambient light. As mentioned above, a positive first charge and a negative second charge are illustrated in FIGS. 1 and 2A, by way of example. Then, a light 150 is applied to a location 134 on the surface 132 of the insulating photoconductive layer 130, while the remainder of the photoconductive surface 132 is protected from the light 150. When the light 150 is applied to the location 134, as shown in FIG. 2A, the illuminated location 134 becomes electrically conductive during illumination and the second charge on the photoconductive layer surface 132 will be discharged or dissipated through the photoconductive layer 130 to the underlying conductive layer 120 at that location 134. The arrows pointing to the conductive layer 120 in FIG. 2A illustrate the discharging of the negative second charge into the conductive layer 120. The second charge on the remainder of the surface 132 remains unchanged. The charge of the illuminated location 134 of the photoconductive layer 130 changes to or acquires the first charge of the underlying conductive layer 120 during illumination. The arrows pointing to the photoconductive layer 130 within the dashed lines in FIG. 2A illustrate the acquisition of the positive first charge at the surface where illuminated. In this embodiment, the light 150 is collimated or focused light beam.

FIG. 2A illustrates a cross sectional side view of the apparatus 100 when illuminated in a location 134. In effect, the illuminated location 134 is electrically connected to the underlying conductive layer 120 during illumination. The conductive layer 120 electrically influences the illuminated location 134 by providing the first charge that the illuminated location 134 ultimately acquires during illumination. This first charge is different from the charge before illumination and therefore, is different from the second charge on a non-illuminated remainder of the surface 132 surrounding the illuminated location 134.

Once the negative second charge is conducted or discharged through the photoconductive layer 130 to the conductive layer 120, such that the illuminated location 134 acquires the positive first charge at the surface, the light 150 is removed or terminated. The electrical conductivity of the illuminated location 134 returns to being electrically nonconductive when the illumination is terminated. Further, the positive first charge on the conductive layer 120 preferably is removed and/or terminated after or when the illumination is terminated. FIG. 2B illustrates the cross sectional view of the apparatus 100 after illumination of the location 134 from FIG. 2A. After illumination, the photoconductive layer 130 comprises a negative second charge surrounding a positive first charge at the surface 132, and the conductive layer 120 no longer has a positive first charge.

The light 150 can illuminate a plurality of locations either by moving the light to predetermined locations for illumination, or by moving the substrate 110 relative to the light 150 to illuminate the predetermined locations, such as with a computer controlled movable stage. Alternatively, a mask can be applied to the surface 132 of the photoconductive layer 130. The mask protects the surface from the light 150 except for a predetermined pattern of locations formed in the mask. In this alternative embodiment, the substrate and/or the light need not be moved relative to the other to create a plurality of illuminated locations 134. Moreover, the light 150 illuminates all or a subset of the predetermined locations at one time and need not be a focused or collimated beam.

The beam of light 150 is generated by a light source. There are many light sources and illumination techniques that can be used for the invention ranging from those light sources that produce the focused or collimated light beam, such as a laser, to other light sources that produce non-collimated light. The intensity of the light need not be more than is necessary to effect a change in the conductivity of (i.e., activate) the photoconductive layer. Moreover, the wavelength of the light is that which would not be detrimental to the chemical or biochemical materials to be deposited. As mentioned above, when using a non-collimated light source, a negative or a mask layer is used to produce the illuminated image(s). Moreover, a light source having an addressable array of small light-emitting devices (e.g., light emitting diodes (LEDs)) may be utilized to supply the light needed to stimulate the photoconductive effect on the substrate 110. When the array light source is used, it is held with a light-emitting surface thereof in close proximity to the surface 132 of the photoconductive layer 130. The array light source illuminates the photoconductive layer 130 at the predetermined locations 133 corresponding to the desired deposition sites 134. In a preferred embodiment, the array light source has 1 to 500 micron spacing between centers and during illumination, there is 0 to 200 microns or more distance between the light emitting surface and the surface 132 of the photoconductive layer 130. Any of the illumination processes are repeatable until all of the desired deposition sites 134 have had a change in electric charge at the surface 132 for each chemical or biochemical material to be deposited.

FIGS. 2A and 2C illustrate a point or collimated beam of light 150 on the illuminated location 134 with the rest of the surface otherwise being protected from the beam of light 150. A scanning laser can provide a focused and precisely located beam of light 150 to each of the illumination locations 133. A mask or other protective means may be used to block the light from the surrounding or non-illuminated area 136. Typically, a photomask that defines the pattern of predetermined locations is used to block the light from the surface of the photoconductive layer 130 except at locations where a change in charge is desired.

FIG. 2C illustrates a perspective view of the apparatus 100 being illuminated with a collimated beam of light 150 wherein a plurality of locations 133, 134, 135 are in different stages of the process of being created. The conductive layer 120 and the photoconductive layer 130 overlying the conductive layer 120 are illustrated with an exemplary array pattern of illumination locations 133, some of which have been illuminated 134 and others, illustrated as dashed circles 135, are to be illuminated by the light 150. The illuminated locations 134 have the changed or first charge and the non-illuminated surrounding area 136 on the surface 132 has the original second charge (not shown in FIG. 2C). For the example above, the illuminated locations 134 have the positive first charge of the conductive layer 120 and the non-illuminated surface 136 has the negative second charge.

The array pattern illustrated in FIG. 2C exemplifies one of many different regular, spatially addressable patterns that can be formed according to the invention. One skilled in the art is familiar with array patterns, especially those used for biological array fabrication. All such patterns are within the scope of the present invention. The pattern depicted in FIG. 2C is neither to scale in size of the substrate 110 nor in number of the illumination locations 133. Further, the shape of the apparatus 100 illustrated in the Figures can range anywhere from round to square and rectangular to elliptical, for example. Therefore, the illustrated sizes, shapes and numbers are not intended to limit the scope of the invention in any way. In some embodiments, the pattern created on the surface of the apparatus 100 can be created by a pulsed, scanning laser. As stated before, this effect could be produced by any method capable of selectively illuminating regions 133 on the apparatus surface 132.

FIG. 3 illustrates a perspective view of the apparatus 100 with its exemplary array of illumination locations 133, all of which being distinguished or delineated by illumination (locations 134) on the photoconductive layer 130. The illuminated locations 134, also referred to herein as deposition sites or regions, have the first charge that is different from the second charge of the surrounding non-illuminated surface area 136. The apparatus 100 effectively comprises an electric charge differential on the surface 132 of the photoconductive layer 130. In accordance with the invention, the apparatus 100 electrostatically guides ionized droplets of the material to be deposited to either the deposition site 134 or the surrounding non-illuminated remainder of the surface 136 using an electrostatic force. The ionized droplets have a charge that facilitates the electrostatic guidance of the droplets. One or both of an attractive force between opposite or different charges and a repulsive forces between the same or less different charges may be employed to guide the ionized droplets during chemical array fabrication according to the invention. For example, the ionized droplets are ionized with a charge that is opposite to or different from the first charge on the deposition sites 134, when the material of the ionized droplets is to be deposited on the deposition sites 134. Alternatively or additionally, the charge of the ionized droplets is the same as or similar to the second charge on the non-illuminated surrounding area 136 when the material is to be deposited on the deposition sites 134. Since opposite charges attract and same charges repel, the ionized droplets will more likely, and advantageously, more accurately be placed on the deposition sites 134 of different charge instead of the non-illuminated surrounding area 136 of similar charge on the surface 132.

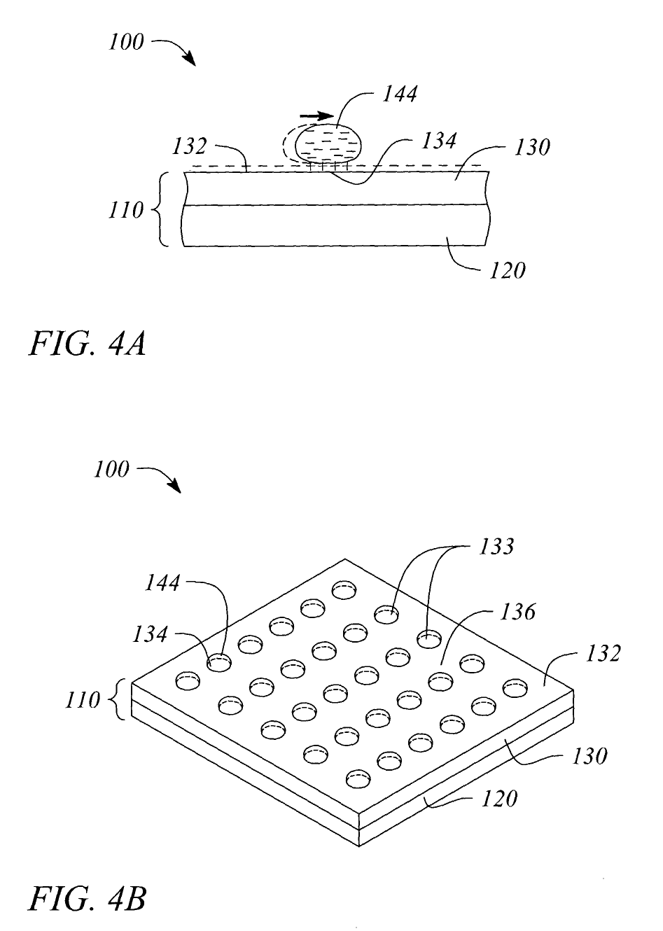

The ionized droplets essentially are guided into position by their inherent attraction to the differently charged deposition sites 134 and their inherent repulsion from the similarly charged non-illuminated surrounding surface 136. FIG. 4A illustrates a cross sectional side view of the apparatus 100 having an ionized droplet 144 with a negative charge on the surface 132 of the photoconductive layer 130, wherein the ionized droplet 144 is more accurately positioned over the differently charged deposition site 134 than if no charges existed. By more accurate, it is meant that the ionized droplet 144 overlaps the illuminated location 134 consistently more than the overlap attained without the present invention. The dashed outline of the ionized droplet 144 is provided in FIG. 4A to exemplify a less accurate position and overlap of the droplet 144 without the benefit of the present invention. The directional arrow emphasizes that the present invention improves position accuracy.

The present invention works particularly well using both attractive and repulsive forces together to guide the ionized droplets into charged desired locations on the surface 132 when the undesired locations are also charged.

It should be noted that the present invention works well for using a repulsive electrostatic force alone to guide the ionized droplets into uncharged or neutrally charged desired locations on the surface 132 when undesired locations are charged. As such, using just a repulsive electrostatic force, the second charge alone on the non-illuminated area 136 can guide similarly charged ionized droplets into deposition sites 134 having a zero potential or neutral charge, such as when the conductive layer 120 is attached to ground during the illumination process. Likewise, the first charge alone on the deposition sites 134 can guide similarly charged ionized droplets into the non-illuminated area 136 having no charge, such as when an electrostatic charge is not applied to the surface 132.

Moreover, the present invention also works well using an attractive electrostatic force alone to guide the ionized droplets into charged desired locations on the surface 132 when the undesired locations on the surface 132 are not charged or are neutrally charged. As such, the second charge alone on the non-illuminated area 136 can guide differently charged ionized droplets to the non-illuminated area 136, when the deposition sites are neutrally charged. Likewise, the first charge alone on the deposition sites 134 can guide differently charged ionized droplets into the deposition sites 134, when the non-illuminated area 136 is neutrally charged.

The electric charge differential created on the surface 132 by the apparatus 100 effectively acts as a deposition or pattern mask for the application of different materials or the same material at different times to the substrate 110. Therefore, after a first material is applied to a first created electric charge differential mask pattern, the charge pattern can be erased, changed or reapplied advantageously without the application of physical materials or requiring a chemical process to do so. For example, a second material is deposited on a second selected set of deposition sites 134 that comprises fewer or more deposition sites 134, having the same and/or different deposition sites than those that received the first material. A second charge differential pattern mask is created by reapplying the second charge to the entire surface 132, illuminating the deposition sites 134 of the second selected set with light, as described above, to change the charge at the selected illuminated locations 134 to the first charge, and depositing ionized droplets of the second material on the substrate 110 having the second electric charge differential pattern mask on the surface 132. This procedure is repeated for each material to be deposited and each incidence that a same material is deposited.

Each selected set of deposition sites 134 comprises a presently illuminated region 134. A selected set may further comprise a previously illuminated region 134 that is presently illuminated and/or a newly illuminated region 135 that was not previously illuminated. As such, each selected set may comprise the same or different sites 134 from other selected sets and may differ by one or more deposition sites 134. FIG. 4B illustrates a perspective view of the apparatus 100 having a plurality of ionized droplets 144 of a material deposited on the illuminated regions 134 of the photoconductive layer 130 using the electrostatic guidance of the invention according to any one or more of the embodiments of the apparatus 100 described above.

As mentioned above, the apparatus 100 of the present invention can also electrostatically guide a material to the surrounding surface 136 instead of the deposition site 135. In some embodiments, a layer of material on the surrounding surface area 136 may be desired. Droplets of the material can be ionized and applied to the surface 132 to electrostatically cover or coat the surface area 136 surrounding the deposition sites 135. This material may provide a reagent or another chemical to the surrounding surface 136 or provide an optical surface, for example. In some embodiments, the material may be coated over the surrounding area 136 to block light therefrom. For an optical surface, the surrounding area 136 may be coated with an opaque or black material layer, for example, to create greater optical contrast that may be useful for subsequent processing of the apparatus 100. As mentioned above, a layer of material deposited on the surrounding surface 136 may be achieved by illuminating the surrounding surface 136 instead of the plurality of deposition sites 135 to change the charge on the surrounding surface 136 for such a deposition.

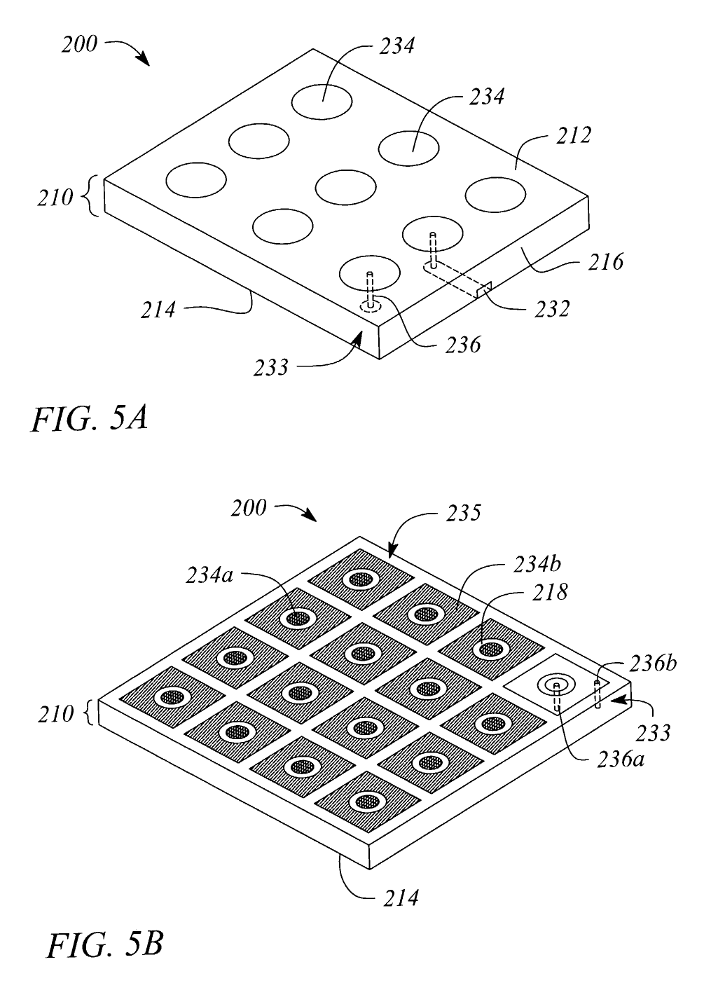

According to other embodiments of the invention, an apparatus 200 comprises a substrate 210 made of a relatively electrically nonconductive or electrically insulative material. The substrate 210 comprises a plurality of deposition sites in a spatially addressable array pattern on an array surface of the apparatus 200, and electrical circuitry 233 including a plurality of electrode pads 234 in an array pattern similar to the pattern of deposition sites, and a plurality of circuit paths 236. The circuitry 233 is made of an electrically conductive material. The conductivity of the circuitry 233 and the substrate is relative, such that semiconductor materials may be used to fabricate the apparatus 200. The plurality of electrode pads 234 either is or corresponds to the plurality of deposition sites. FIG. 5A illustrates an example of the plurality of electrode pads 234 on or just under a surface 212 of the substrate 210. The electrode pads 234 are electrically isolated from one another by the nonconductive substrate material. The plurality of electrically conductive circuit paths 236 is connected to the electrode pads 234 to provide controlled electrical access to the electrode pads 234. The circuit paths 236 permit controlled charges to be established separately or independently on each electrode pad 234. The circuit paths 236 are embedded in the substrate 210. FIG. 5A illustrates only some circuit paths 236 that are possible by dashed-lines through the substrate 210 in FIG. 5A.

FIG. 5B illustrates another example of more complex electrode design for the apparatus 200. In this example, each deposition site 235 either comprises or corresponds to at least two individually controlled electrode pads 234a and 234b that are electrically isolated from one another by the relatively nonconductive substrate material, as illustrated at the substrate surface 218 within each site 235. The electrode pads 234a, 234b at each site 235 are individually electrically connected to respective circuit paths 236a, 236b, such that individual control of the deposition of the ionized droplets or aerosol particles of a material at each site 235 is achieved. FIG. 5B illustrates the circuit paths 236a, 236b as dashed lines through the substrate 210 by way of example. The electrode shading is removed from one of the sites 235 to better illustrate the embedded circuit paths 236a, 236b. It may be advantageous and is within the scope of the present invention to use more than two electrode pads per deposition site 235, for control of droplet trajectories as the droplets or particles approach the electrodes.

The number and shape of the electrode pads 234, 234a, 234b are not restricted to the examples shown in FIGS. 5A and 5B. Some or all of the electrical circuitry 233 could be located on the substrate surface 212 and be optionally covered with an insulating material (not shown in FIGS. 5A and 5B). In addition or alternatively, some or all of the electrical circuitry 233 may be embedded in the substrate 210 (the circuit paths 236, 236a, 236b are shown embedded in FIGS. 5A and 5B by way of example). The electrode pads 234, 234a, 234b may be one or more of on the surface 212 and in or just underneath the surface 212 (i.e., ‘at’ or ‘adjacent to’ the surface 212). A variety of technologies and materials that are well known in the art of thick film, thin film and semiconductor circuit fabrication may be used to fabricate the electrical circuitry 233 and substrate 210 for the apparatus 200. As such, the apparatus 200 may comprise a multilayer substrate 210 depending on the electrical circuitry pattern and fabrication implementation used. The materials used for the apparatus 200 also include those materials classified as semi-conductive materials.

Suitable circuitry terminations are provided at a convenient location on the substrate 210 to connect to off-substrate electronics or electronic circuitry (not shown). FIG. 5A illustrates one example of a termination 232 provided along an edge 216 of the substrate 210. In other embodiments, the terminations (not illustrated) may be on an opposite surface 214 from the surface 212, as illustrated in by the dashed lines in FIGS. 5A and 5B. The apparatus 200 embodiments illustrated in FIGS. 5A and 5B are not to scale and are exemplary only. It is within the scope of the invention for the shape of the substrate 210 to be any shape from round to square and rectangular to elliptical. Further, the pattern and number of deposition electrode pads 234, 234a, 234b illustrated are exemplary only. For example, the array pattern could be annular, having a circular pattern or spiral pattern, for example, instead of a series of rows and columns. The shape of the electrode pads 234, 234a, 234b can be any shape ranging from square to rectangular, to elliptical or to circular. Also, the substrate 210 may be part of a larger wafer having a plurality of arrays thereon, or may be the wafer itself (not shown). The wafer is diced into individual substrates 210, each having an array of the plurality, according to techniques that are well known in the art. The number, size and shape of the deposition electrode pads 234, 234a, 234b are limited by the fabrication technology used, and therefore, are not intended as a limitation to the present invention.

The electrical circuitry 233 connects to off-substrate control electronics or electronic circuitry, such as a voltage or current source, to provide a charge and/or a ground connection to the electrode pads 234, 234a, 234b. The circuit paths 236, 236a, 236b supply the first charge to the electrode pads to accrete the material to, or to repel the material from, the deposition sites electrically influenced by the charged electrode pads. When a set or selection of pads of the plurality of electrode pads 234, 234a, 234b are charged with the first charge, ionized droplets can be deposited onto substrate surface 212 at each deposition site either directly or indirectly corresponding to the charged set of electrode pads.

For the purposes of the invention, a deposition site directly corresponds to an electrode pad when the electrode pad functions as, or essentially is, the deposition site, such that the first charge on the electrode pad is a charge on the deposition site. A deposition site indirectly corresponds to an electrode pad when there is some degree of separation between the electrode pad and the respective deposition site and the first charge on the electrode pad electrically influences the respective deposition pad with a corresponding electric field, as is further described below with respect to apparatus 300. For example, using different materials for the electrode pad and the deposition site and/or providing a physical space between the electrode pad and the deposition site may achieve the degree of separation.

Each selected set of electrode pads comprises either one particular electrode pad, all of the electrode pads of the plurality, or any number of the same and/or different electrode pads therebetween. The ionized droplets of a first material have a charge that facilitates the electrostatic guidance of the first material for chemical array fabrication. The ionized droplets will be electrostatically guided to the deposition sites corresponding to or electrically influenced by a first set of charged electrode pads, while no preferential guidance is provided to any uncharged electrode pads or other surfaces. After the first material is deposited on respective deposition sites corresponding to the first selected set of charged electrode pads, the charge is removed. The deposited material chemically bonds to the deposition site surface, so that the removal of the charge does not impact the deposited material.

A second material is deposited by applying another first charge to a second selected set of pads 234, 234a, 234b. Some of the electrode pads of this second set may be the same as the previously charged electrode pads of the first set for the deposition of the first material. Other electrode pads of the second set may not have been previously charged for receiving the first material. In this way, a variety of different materials can be deposited on deposition sites corresponding to a variety of different sets of charged electrode pads sequentially, until each electrode pad 234, 234a, 234b or corresponding deposition site comprises a desired chemical composition of one or more deposited materials.

Further, the apparatus 200 can control the volume of each deposited material on each deposition site with the charge that is applied (or the length of time that the charge is applied) to each electrode pad 234, 234a, 234b of a set for each material. Accordingly, array fabrication materials are more precisely placed in desired locations in more precise volumes using the present invention than for an array without the present invention. The apparatus 200 effectively creates a ‘soft’ deposition mask having a changeable pattern mask with different electric charge or field differentials created at the array surface. The deposition mask can be changed with each different application of charge to different sets of electrode pads 234, 234a, 234b.

In some cases, it may be desirable for the substrate 210 and/or at least some of the electrical circuitry 233 to be transparent, when the apparatus 200 is used for a DNA/RNA biological microarray assay, for example. Transparent substrate materials used for biological array manufacture are well known in the art. A common transparent substrate is transparent to optical scanning, such as with the laser. Optically transparent materials used for the electrical circuitry 233 also are known in the art, such as the materials used in display technology, for example. Accordingly, the apparatus 200 can be manufactured using well-known optical transparent substrate materials and using optical transparent electrically conducting materials.

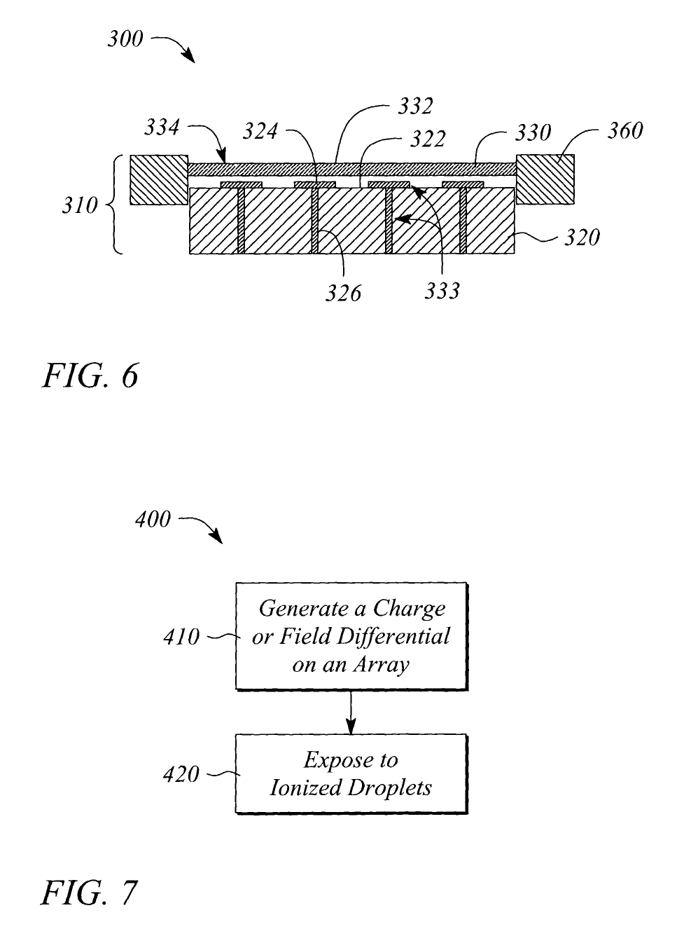

According to still other embodiments, an apparatus 300 can be used in the fabrication of biochemical arrays, such as DNA/RNA microarrays, without regard to transparent circuitry. FIG. 6 illustrates a cross sectional side view of an embodiment of the apparatus 300 comprising a substrate 310 that comprises a membrane sheet 330, a plurality of deposition sites on an array surface 332 of the membrane sheet 330, a plate 320 underlying the membrane sheet 330, and electrical circuitry 333. In these embodiments, the electrical circuitry 333 comprises electrically conductive electrode pads 324 and electrically conductive circuit paths 326 that are embedded in and/or provided on or adjacent to a surface 322 of the plate 320, in much the same way as described above for apparatus 200. Preferably, the plate 320 is similar or equivalent to the substrate 210 described above. The electrode pads 324 are arranged in an array pattern on or in the surface 322 of the plate 320. A circuit path 326 provides a first charge to a respective electrode pad 324. The membrane sheet 330 is adjacent to the surface 322 of the plate 320 where the electrode pads 324 are located. The charge on the electrode pads 324 of the underlying plate 320 generates an electric field through the membrane sheet 330 at deposition locations or sites 334 on the array surface 332 of the membrane sheet 330 corresponding to the locations of the underlying electrode pads 324. Therefore, the deposition sites 334 indirectly correspond to the electrode pads 324 and the circuitry 333 of the apparatus 300 electrically influences the deposition sites 334 on the membrane sheet 330 with the generated electric field.

According to the invention, a material can be deposited on a selected set of the deposition locations or sites 334 of the membrane sheet 330 by charging a corresponding set of the underlying electrode pads 324 and introducing ionized droplets of the material to the array surface of the membrane 330. The ionized droplets of the material have a charge that facilitates electrostatic guidance of the material for chemical array fabrication. An electrostatic force between the charge of the ionized droplets and the electric field of the corresponding electrically influenced deposition sites 334 guide the ionized droplets of the material to the influenced deposition sites 334 or a remaining array surface 332 surrounding the influenced deposition sites 334 depending on whether the electrostatic force is one or both of an attractive force and a repulsive force.

Advantageously, the material is deposited more precisely on the deposition sites 334 of apparatus 300, for example, than without the present invention. Other materials may be deposited on the same set or other sets of deposition sites 334 or remaining surrounding surface to a final composition at each deposition site 334 and/or on the array surface in much the same way as described above for the apparatus 200. Moreover, electronic control circuitry (not illustrated in FIG. 6) can vary the charge provided to one or more charged electrode pads 324 to increase or decrease ongoing accretion of ionized droplets to the deposition site 334 on membrane sheet 330 corresponding to the charged electrode pad 324 during a deposition process. Advantageously, the apparatus 300 overcomes any obstacles to providing transparent circuitry to the substrate, as might be considered when using some embodiments of the apparatus 200. Further, the apparatus 300 essentially eliminates any compatibility issues between the material to be deposited and the material of the electrode pads, also as might be considered for some embodiments of the apparatus 200.

The membrane sheet 330 of the apparatus 300 is made from flexible membrane substrate materials that are commonly used for biological array manufacture and that are well known in the art. Due to the flexibility of the membrane material, the membrane sheet 330 may be mounted to a membrane support 360, such as a support frame, to provide mechanical stability to the membrane sheet 330. After the array of materials are deposited on the membrane 330, the membrane 330 can be removed from the plate 320 for further processing, such as a hybridization assay of the deposited material with a target sample, for example, or other assay, and a subsequent optical interrogation process. Both of the apparatus embodiments 100 and 200 also can be further processed for hybridization or other assays, and subsequently interrogated by a variety of means known in the art.

The present invention also provides a method of electrostatically guiding a chemical or biochemical material to be deposited in chemical array fabrication. FIG. 7 illustrates a method 400 that comprises generating 410 an electric charge or field differential on an array; and exposing 420 the array to an ionized droplet of the chemical or biochemical material to be deposited. The ionized droplet has a charge that facilitates electrostatic guidance according to the method 400. As described above, the charge of the ionized droplets is different from the charge or field influencing a surface location on which the ionized droplet is to be deposited, such that the material preferentially deposits at the influenced surface location. Otherwise, the charge of the ionized droplets is similar to the charge or field influencing a surface location, such that the material preferentially deposits at locations other than the influenced surface location.

The array comprises a plurality of deposition sites in a spatially addressable array pattern on an array surface. The electric charge or field differential comprises a first charge that electrically influences a selected set of the deposition sites of the plurality, and a second charge on an area surrounding the selected set. The first charge is different from the second charge. Preferably, the array is any embodiment of the apparatus 100, 200, 300 described above.

The electric charge or field differential may be generated 410 on the array in several ways. The electric charge or field differential is generated 410 by applying or supplying the first charge to regions on the array that either directly or indirectly correspond to the selected set of deposition sites. According to some embodiments, as described above for the apparatus 100, generating the electric charge or field differential comprises providing an array substrate that comprises a layer of an electrically nonconductive photoconductive material over an underlying electrically conductive layer. In these embodiments, generating an electric charge or field differential further comprises applying the second charge to a surface of the photoconductive layer, while the photoconductive layer is protected from exposure to light. Generating an electric charge or field differential further comprises connecting the conductive layer to a first charge source or sink; and illuminating unprotected regions of the electrostatically charged surface of the photoconductive layer with light. The unprotected regions correspond to the selected set of deposition sites or to the surrounding area. The illuminated regions become electrically conductive while being illuminated and dissipate or discharge the second charge on the photoconductive layer surface at the illuminated region to the conductive layer. The illuminated region acquires the first charge of the underlying conductive layer also while being illuminated. The illumination is momentary, such that the charge at the illuminated location is changed from the second charge to the first charge, while the second charge at a non-illuminated remaining portion of the photoconductive layer surface is unchanged. As a result, an electric charge differential is generated at the array surface.

According to other embodiments of the method, generating an electric charge or field differential comprises providing an array substrate, such as that described above for the apparatus 200. The array substrate is made of an electrically nonconductive material that comprises the plurality of deposition sites and further comprises electrical circuitry. The electrical circuitry comprises a plurality of electrically conductive electrode pads connected to electrically conductive circuit paths that are embedded in the substrate. The plurality of electrode pads either directly or indirectly corresponds to the plurality of deposition sites. Preferably, the circuit paths are embedded in the substrate and the electrode pads are on or in the substrate adjacent to the array surface. Generating an electric charge or field differential further comprises applying the first charge to the circuit paths that interconnect to a selected set of the electrode pads to charge the selected set of electrode pads. The selected set of electrode pads correspond to the selected set of influenced deposition sites. The surrounding area is a portion of the array surface not influenced by the first charge.

According to still other embodiments of the method 400, generating an electric charge or field differential comprises providing an array substrate, such as the apparatus 300 described above. The array substrate comprises an electrically nonconductive membrane sheet overlying a plate of an electrically nonconductive material having electrically conductive circuitry embedded therein and/or disposed thereon. The plurality of deposition sites are on the membrane sheet, such that the membrane sheet provides the array surface. The circuitry comprises electrically conductive circuit paths connected to electrically conductive electrode pads. Each deposition site on the membrane sheet has an indirectly corresponding electrode pad in the plate. The electric charge or field differential is generated according to the other embodiments of the method 400 by applying the first charge to a selected set of electrode pads of the plate corresponding to the selected set of deposition sites on the membrane sheet. The first charge on the selected set of electrode pads creates an electric field through the membrane sheet at each deposition site of the selected set of deposition sites indirectly corresponding to the charged electrode pads.

For the purposes of the method 400, the selected set of deposition sites may be any desired deposition sites ranging from one particular site to all of the plurality of sites. Each selected set may be the same or different from previously selected sets. According to some of the embodiments using the apparatus 200 or 300, either same or different sets of electrode pads may be charged to influence respective same or different sets of deposition sites during chemical array fabrication to deposit a plurality of chemical or biochemical materials on the array surface until desired compositions of materials are achieved at each deposition site.

Further according to the method 400, the array is exposed 420 to ionized droplets of the material to be deposited in several ways according to the invention. Exposing 420 the array to ionized droplets comprises forming ionized droplets of the material and dispensing the ionized droplets to the array. Droplets of the material are formed using droplet generators. A droplet generator is any piece of equipment that produces droplets of the material composition. In some embodiments, the droplet generator is any one of a variety of print heads, including, but not limited to ink-jet print heads. Ink-jet deposition equipment has characteristics, such as defined or controlled droplet positioning and defined droplet volume, which makes this technology quite attractive for chemical array manufacture. In other embodiments, the equipment includes, but is not limited to, sprayers and aerosol generators (also known as fog generators or foggers) that generate microdroplets and that are designed to provide a blanket of coverage to a surface rather than defined positioning of droplets. All of such equipment for generating droplets of a material composition are well known in the art and are within the scope of the present invention.

The material composition may be charged or ionized prior to loading the composition into the droplet generation equipment, charged by or in the equipment, charged as the equipment generates the droplets, or otherwise, using well-known methods. Any method or technique used to charge or ionize droplets is within the scope of the present invention.

Ink-jet technology can be used to deposit a plurality of different material compositions selectively on the array surface in a single layer during the step of exposing 420 the array to ionized droplets. The ionized droplets are fired from an ink-jet print head to the surface of the array at predetermined locations. The ink-jet print head directs individual ionized droplets to electrically influenced deposition sites. While the ink-jet technology has relatively precise positioning compared to other droplet generators, array manufacturers strive to improve the precision. Improved precision allows for more densely packed array patterns to be manufactured on a single substrate. Further, improved precision minimizes errors in droplet placement and improves results of biological array assays of a target material under test. Target samples typically are available in only very minute quantities and are expensive to obtain. More densely packed arrays use less of the target sample in an assay. Further, more accurate assay results minimize target sample waste. Advantageously, the present invention enhances the precision of ink-jet deposition equipment by rendering the predetermined locations with a different charge than the rest of the surface and electrostatically guiding the fired ionized droplets having a charge to the differently charged predetermined locations using one or both of attractive and repulsive forces, for example.

Aerosol generators or foggers also can be used to form ionized droplets of, and to separately dispense, a variety of different chemical compositions on the differently charged surface of an array according to the invention. Chemical species to be deposited are first converted into aerosols of electrically charged microdroplets. Preparation of aerosols is a well-known process; see for example Hinds, William C., Aerosol Technolog, Wiley Inc., 2^{nd} Edition, January 1999, which is incorporated herein by reference. An aerosol is generally taken to mean a suspension of solid or liquid particles in a continuous gas phase. However, the present invention is useful also where the continuous phase is a rarified gas, a dense gas, an immiscible liquid, or a partially miscible liquid. The immiscible liquid and partially miscible liquid are usually known as emulsions. All suitable forms of these mixtures are within the scope of the present invention. “Suitable” refers to the continuous phase being an insulator, and the dispersed phase being ionized.

The microdroplets from the aerosol generator are dispensed as a blanket or a fog over the differently charged array surface. When a sprayer or aerosol generator is used to generate droplets of a material, exposing 420 the array to ionized droplets comprises either moving the array through the fog of charged microdroplets or moving the fog of microdroplets over the array. The charged microdroplets in the fog are either attracted to or repelled by the electrically influenced deposition sites of the array due to electrostatic attractive or repulsive forces, and in some embodiments, the charged microdroplets are guided by both attractive and repulsive forces. As such, charged microdroplets from the fog can be deposited to synthesize polymers or biopolymers on the influenced deposition sites. Further, a fog of a material can be deposited to buildup into a continuous layer on the area surrounding the deposition sites. To build-up a continuous layer on the surrounding area, the charge of ionized droplets and on the electrically influenced deposition sites are rendered the same or similar so that the microdroplets are repelled therefrom to the surrounding area. Alternatively or additionally, an electrostatic charge can be applied to the surface of the surrounding area that is different from the electrically influenced deposition sites, such that the charged ionized droplets are further attracted to the surrounding area.

In accordance with the method 400, both generating 410 an electric charge or field differential on an array and exposing 420 the array to ionized droplets of a material are repeated for each different set of deposition sites, each continuous layer applied to the surrounding area, each different material to be deposited, and for each different application of the same material. In this way, material layers can be built up on the surface of the array by creating a series of electric charge or field differential deposition masks and coating selected regions on each successive layer. Over a number of cycles, numerous small areas on the substrate (e.g., ultimately probes) can be built up in many layers where the position and size of the application area is controlled by light-produced different charges (as in apparatus 100). Moreover, the charge provided to the electrode pads can be varied to increase or decrease ongoing accretion of ionized droplets to the corresponding deposition sites during a deposition process (as in apparatus 200 and 300). As such, the volume of material deposited is controlled by the electric charge that is applied to the circuitry with the control electronics. For the invention, it is possible to vary accretion of the material on a number of deposition sites ranging from one to all sites, depending on the complexity of the circuitry and the control electronics employed. As such, the present method 400 optionally further comprises varying the first charge that influences a deposition site of the selected set of sites on the array to control accretion of the material on the selected deposition sites.

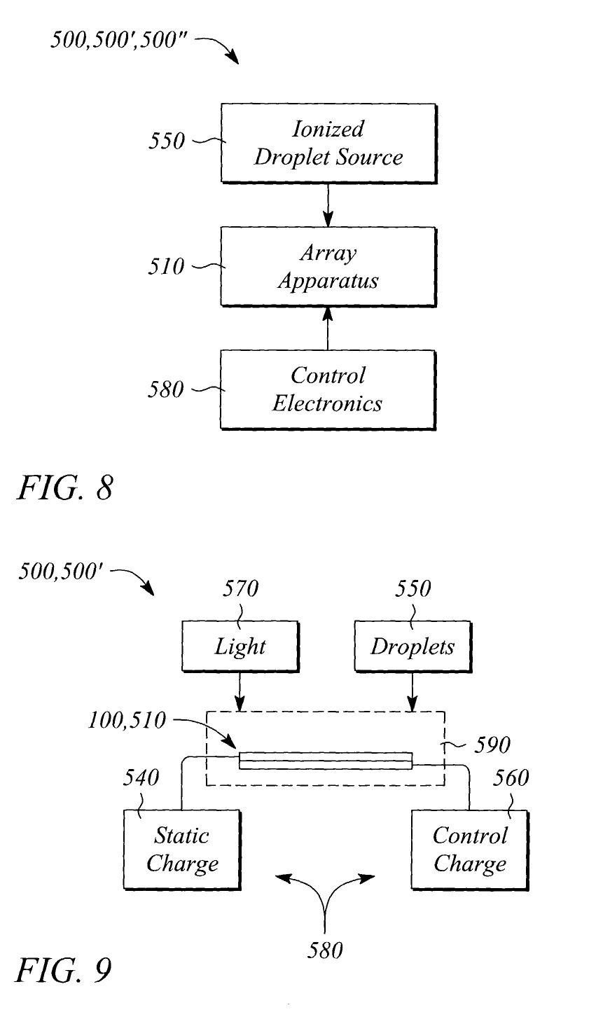

A system 500 for electrostatically guiding ionized droplets of a chemical or biochemical material in chemical array fabrication is provided. The system 500 comprises an array apparatus 510, and a source of ionized droplets 550. The array apparatus can be any embodiment of the apparatus 100, 200, 300. The source of ionized droplets 550 can be any of the droplet generating means described above. FIG. 8 illustrates an embodiment of the system 500. The system 500 may further comprise control electronics 580 that one or more of supplies, removes and varies a charge to the array apparatus 510. Depending on the embodiment, the control electronics 580 comprise one or both of means for generating a controllable charge (i.e., a source of charge) to electrically conductive material, and means for generating an electrostatic charge to a surface, such as to the surface of a nonconductive material. The controllable charge generating means is preferably controllable by being adjustable. In some embodiments, the control electronics 580 further comprises charge-sensing circuitry that controls accretion volumes of the ionized material on the array apparatus, as is further described below.

When the apparatus 100 is employed as the array apparatus 510, the system 500′ further comprises a light source 570. The control electronics 580 comprise both the means 540 for electrostatic charge generation and the means 560 for controllable charge generation. FIG. 9 illustrates a block diagram of the system 500′ according to some embodiments. As mentioned above with respect to apparatus 100, the electrostatic charge generating means 540 can be a corona wire, for example. The controllable charge generating means can be a ground connection and/or a voltage source or a current source, as are known in the art. The voltage source and the current source each provides a selection of positive and negative charges, as well as a zero volts or zero amps selection, respectively. The light source 570 can be a laser, such as a scanning laser, or other collimated light source, or a non-collimated light source, for example. In some embodiments of the system 500′, the system 500′ optionally further comprises means 590 for blocking ambient light from the photoconductive layer during chemical array fabrication. The means 590 for blocking ambient light protects the surface from the light, and can range from a light-blocking mask positioned over the photoconductive layer to a light blocking enclosure to house the apparatus 100, and further to a dark room, such as that used in photography. Not illustrated in FIG. 9 is that the system 500′ optionally further comprises a movable stage that one or more of moves the apparatus 510 to align with the light source 570 and/or the ionized droplet generator 550 and moves the light source 570 and/or the droplet generator 550 to align with the apparatus 510. The movable stage facilitates automation of chemical array fabrication using the system 500′.

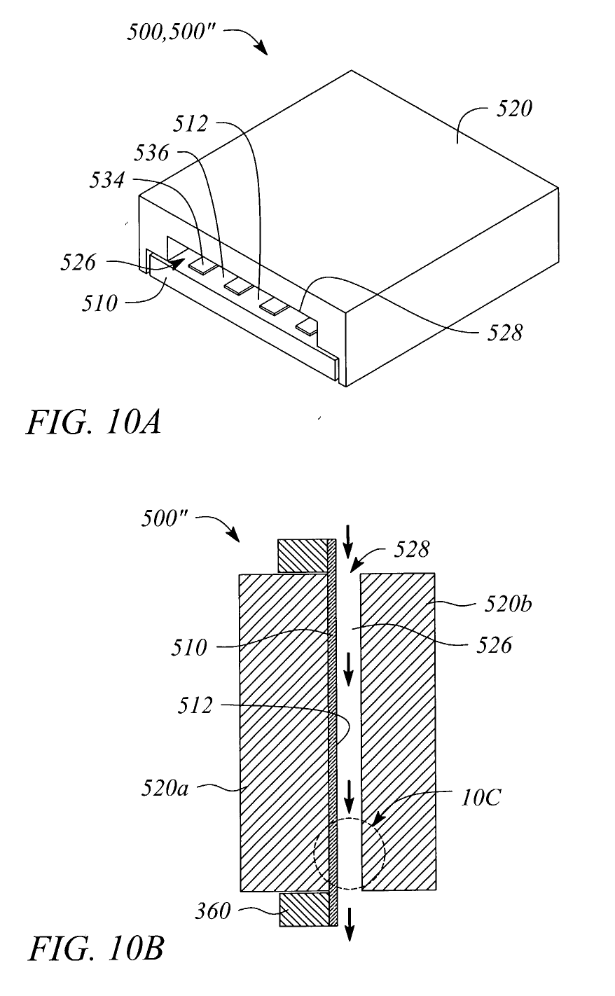

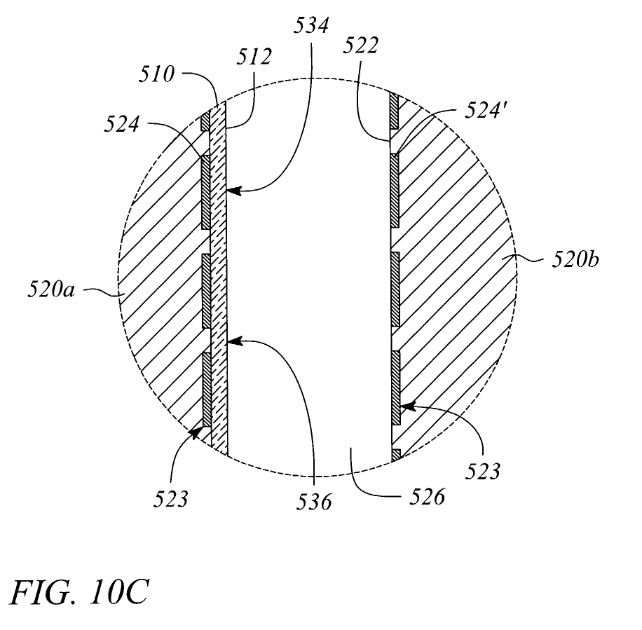

When apparatus 200 or 300 is employed as the apparatus 510, the system 500″ further comprises a cell or chamber 520 that encloses the array 510. FIG. 10A illustrates a perspective view of the system 500″, according to some embodiments. Not illustrated in FIG. 10A are the ionized droplet source 550 and the control electronics 580 for simplicity purposes only. The cell 520 comprises a volume space 526 adjacent to deposition sites 534 of the array apparatus 510, and a port 528. In some embodiments, the cell 520 encloses the surface 512 of the array 510. In other embodiments, the cell encloses at least the surface 512 and preferably, more of the array apparatus 510, such as enclosing all of the apparatus 510.

FIG. 10B illustrates a cross sectional view of an embodiment of the system 500″ using an embodiment of the apparatus 300 as the array apparatus 510. In this embodiment of the system 500″, the plate 320 of apparatus 300 functions as a part (e.g., substrate portion 520a) of the cell 520. The membrane sheet 330 of the apparatus 300 is the apparatus 510, as described herein. The cell 520 comprises a substrate portion 520a underlying the membrane sheet 510. The substrate portion 520a comprises circuitry 523 (not illustrated in FIG. 10B) that electrically influences deposition sites 534 on the membrane sheet 510. Also, the array membrane 510 is supported by the frame support 360, which was described above for the apparatus 300. FIG. 10C is a magnified view of a section labeled 10C of the embodiment illustrated in FIG. 10B. The magnified view in FIG. 10C illustrates the cell circuitry 523 having electrodes 524. The electrodes 524 are electrically connected to circuit paths (not illustrated) that ultimately electrically connect to the control electronics 580 (also not illustrated in FIG. 10C).

FIG. 10C further illustrates that the cell 520 may further comprise an array pattern of electrodes 524′ on an interior surface 522 of a cell portion 520b spaced from the array membrane 510 and facing the deposition sites 534 of the array membrane 510, according to some embodiments. The electrodes 524′ are offset from the array membrane 510 surface 512 by a short distance defined by the volume 526 of the cell 520. Charges on the electrodes 524′ also influence the motion of microdroplets, thereby assisting in control of droplet trajectories. Therefore the electrodes 524′ of the cell portion 520b may be considered to be a part of the cell circuitry 523.

An aerosol of a chemical composition to be deposited on the array membrane 510 flows from the ionized droplet source 550, which is an aerosol generator for this embodiment, into the cell 520 through port 528 to fill the volume 526, as illustrated with bold arrows in FIG. 10B, for example. The deposition sites 534 (equivalent to sites 334 of the apparatus 300) on the array membrane 510 are electrically influenced by a first charge on the electrode pads 524 of the cell 520 that underlie the corresponding deposition sites 534. Intervening areas 536 of the array membrane 510 surface 512 have an electrostatic charge that is different from the first charge, which may be a neutral charge or a non-neutral charge, depending on the embodiment. The aerosol is ionized with a charge that different from the first charge influencing the array deposition sites 534 so that the ionized droplets are guided to and deposit on the charge-influenced deposition sites 534. The microdroplets of the aerosol are typically carried in the gas phase. The choice of material for the gas phase of the aerosol includes, but is not limited to, air, an inert gas, and a chemically active gas. The gas phase may be at typical atmospheric pressure, rarefied pressure, or dense pressure. An immiscible or partially miscible liquid may also be used as the carrier phase, as mentioned above.

An aspect of the invention is the ability to control accretion at each site by monitoring and controlling charge, in particular with respect to apparatus 200, 300 and system 500, 500″. Thus, the control electronics 580 of the system 500″ optionally further comprise the charge sensing and controlling circuitry, as mentioned above with respect to FIG. 8. As cumulative droplet accretion at a deposition site 534 builds up, control circuitry adjusts or reverses the charge at the electrode pad(s) 524, 524′, thereby adjusting or reversing the electric field influencing the corresponding deposition sites 534. As such, the ionized droplets are repelled and prevented from further accretion at the corresponding deposition site 534. The charge sensing and controlling circuitry controls the potential at each deposition site 524 to allow buildup of the desired deposition volume of each chemical species or material to be deposited. The end result effectively is a ‘soft’ deposition mask, having a changeable electric charge or field differential pattern that also has a changeable charge potential/polarity pattern. Advantageously, the same droplet generator equipment 550, deposition cell 520 and array 510 are capable of generating any desired patterns of microarrays using the charge sensing and controlling circuitry. The charge sensing and controlling circuitry that may be used for the present invention is similar to the charge coupling circuitry commonly used in charge-coupled device (CCD) cameras and is known to one skilled in the art.

The system 500″ for depositing chemical species eliminates using moving print heads for deposition, and eliminates using robots or movable stages for physical transfer of array substrates between process stations. As such, the system 500″ may have no mechanical moving parts. However, in some embodiments of the system 500″, it may be advantageous to move the deposition cell 520 between successive aerosol sources 550 for chemical array fabrication. The system 500″ is amenable to automation such that one or several cells 520 may be connected with multiple aerosol sources 550 and other reagents or process sources, either simultaneously or sequentially.

In situ synthesis of DNA and RNA microarrays is one example of a process where an array substrate is exposed to multiple chemical species sequentially. The embodiments 500′, 500″ of the system 500 are adapted to flood the array 510 (e.g., in aqueous washing, gas-drying, common chemical steps, etc.) when all deposition sites 534 must be treated in the same manner. While reagents and washes typically are flooded over the substrate surface during in situ synthesis, the different nucleotide species are precisely deposited in desired locations to achieve a variety of different nucleotide sequences (e.g. as probes) on the array. The system 500 is amenable to many known automation techniques that would efficiently connect the fabrication steps for simultaneous or sequential processing, and further connect the fabricated chemical array to an assay process, such that the apparatus 510 is not manually handled between fabrication and assay processes.

The system 500″ controls the volume 526 of the cell 520 occupied by the aerosol as it moves over the array surface 512. For example, the volume 526 can be made very small, and environmental factors, such as pressure, temperature and other factors can be controlled and manipulated. Furthermore, in some embodiments, the port 528 is a narrow slot in the cell 520 in communication with the volume space 526. Advantageously, the narrow slot 526 constitutes a Hele Shaw cell, in which aerosol flow is even and free of turbulence, as is well known to one skilled in the art. See for example, Horace Lamb, Hydrodynamics, Sixth Ed., Dover Publications, N.Y., 1936, p. 86 and p. 582, which is incorporated by reference herein. Turbulence has an attendant risk of causing uncontrolled deposition. Therefore, the system 500″ advantageously can control turbulence in the volume space 526 also.

Thus, there have been described embodiments of a novel apparatus, a method and a system for electrostatically guiding a material in chemical array manufacture. It should be understood that the above-described embodiments are merely illustrative of the some of the many specific embodiments that represent the principles of the present invention. Clearly, those skilled in the art can readily devise numerous other arrangements without departing from the scope of the present invention.

What is claimed is:

1. An apparatus that electrostatically guides a chemical or biochemical material to be deposited in chemical array fabrication comprising:
a substrate comprising a photoconductive layer overlying an electrically conductive layer; and
an electric charge differential generated on a surface of the photoconductive layer by selectively illuminating the surface with light, such that the electric charge differential guides a droplet of the chemical or biochemical material having a charge to preferentially deposit on a location of the surface due to an electrostatic force.

2. The apparatus of claim 1, further comprising an array pattern of locations defined on the surface of the photoconductive layer, the array pattern of locations comprising a plurality of spatially arranged deposition sites and an area surrounding the plurality of deposition sites, an illuminated location of the surface being one of either a deposition site of the plurality or the surrounding area, a non-illuminated location of the surface being a different one of either the deposition site of the plurality or the surrounding area.

3. The apparatus of claim 2, wherein the charge of droplet facilitates whether the charged droplet will preferentially deposit on the illuminated location or the non-illuminated location of the surface.

4. The apparatus of claim 2, wherein the plurality of deposition sites supports a polymer synthesized from the chemical or biochemical material, the polymer being used in an assay of a chemical or biochemical sample that uses the apparatus.

5. The apparatus of claim 1, wherein the electric charge differential comprises a first charge on an illuminated location of the surface of the photoconductive layer and a second charge on a non-illuminated location of the surface, the first charge being different from the second charge.

6. The apparatus of claim 5, wherein the substrate further comprises the conductive layer, the conductive layer being electrically connectable a source of charge or a sink of charge to provide the first charge, the illuminated location acquiring the first charge from the conductive layer.

7. The apparatus of claim 1, wherein the photoconductive layer is electrically nonconductive in the absence of light, an illuminated location of the surface of the photoconductive layer being electrically conductive and electrically connected to the conductive layer while being selectively illuminated with light to provide a different electric charge to the illuminated location.

8. The apparatus of claim 7, wherein a non-illuminated location of the surface of the photoconductive layer is electrically nonconductive.

9. The apparatus of claim 1, wherein the electric charge differential comprises one of a positive charge and a negative charge, a positive charge and a neutral charge or ground, a negative charge and a neutral charge or ground, and two different positive charges or two different negative charges on the surface of the photoconductive layer.

10. The apparatus of claim 1, wherein the charged droplet of the material is guided to the location on the surface using one or both of an attractive electrostatic force and a repulsive electrostatic force.

11. A deposition mask having an electrically changeable mask pattern comprising the apparatus of claim 1, wherein the changeable mask pattern comprises a pattern of different electric charges for each selectively illuminated set of locations, a selected set of illuminated locations being provided for each chemical or biochemical material to be deposited and for each incidence that a same chemical or biochemical material is deposited during the chemical array fabrication, each selected set having at least one illuminated location, the at least one illuminated location being a same location or a different location relative to other selected sets.

12. A system for electrostatically guiding the chemical or biochemical material in chemical array fabrication comprising the apparatus of claim 1, and further comprising one or more of:
a light source that provides the selective illumination to the surface;
a source of charge or a sink for charge that provides an electric charge of the differential; and
a droplet generator that provides the charged droplet of the chemical or biochemical material to the apparatus.

13. The system of claim 12, wherein the droplet generator is selected from one or more of an aerosol generator, a fogger or a sprayer of ionized microdroplets of the material and an ink-jet print head that produces individual ionized droplets of the material from a nozzle.

14. The system of claim 12, wherein the source of charge comprises one or more of a corona wire and a voltage or current source, the voltage or current source optionally being adjustable such that zero volts or zero amps is selectable, and wherein the sink for charge is a connection to ground.

15. The system of claim 12, wherein the light source comprises one or both of a collimated beam of light and a non-collimated light beam.

16. An apparatus that electrostatically guides a chemical or biochemical material to be deposited in chemical array fabrication comprising:
a substrate that comprises a plurality of deposition sites spatially arranged in an array pattern on an array surface, and circuitry defined in the substrate that electrically influences the plurality of deposition sites, such that when the circuitry electrically influences a deposition site with a charge, an electric charge or field differential is created at the array surface, the electric charge or field differential guiding an ionized droplet of the material to preferentially deposit on the electrically influenced deposition site or another location on the array surface due to an electrostatic force.

17. The apparatus of claim 16, wherein the circuitry is electrically conductive and connectable to a source of charge, the substrate being electrically nonconductive relative to the circuitry, the circuitry comprising a plurality of circuit paths embedded in the substrate and a plurality of electrode pads on or in the substrate adjacent to the array surface, the plurality of electrode pads directly or indirectly corresponding to the plurality of deposition sites, the plurality of circuit paths providing controlled electrical access to the plurality of electrode pads from the source of charge.

18. The apparatus of claim 17, wherein when the charge is applied to at least one circuit path, at least one electrode pad will be similarly charged, such that the material will accrete either on or around a deposition site corresponding to the charged electrode pad.

19. The apparatus of claim 16, wherein the substrate further comprises an area of the array surface surrounding the plurality of deposition sites, the surrounding area being electrically nonconductive and being chargeable with a surface electrostatic charge that is different from the charge on the circuitry when applied, such that when the surface charge is further applied to the surrounding area, the ionized droplet of the material is guided to the electrically influenced deposition site or the surrounding area due to one or both of an attractive electrostatic force and a repulsive electrostatic force.

20. The apparatus of claim 16, wherein the substrate further comprises a membrane sheet of an electrically nonconductive material, the membrane sheet providing the array surface, the plurality of deposition sites being on the array surface of the membrane sheet.

21. The apparatus of claim 17, wherein the substrate further comprises a membrane sheet of an electrically nonconductive material, the plurality of deposition sites being on the membrane sheet, the membrane sheet providing the array surface, the plurality of electrode pads indirectly corresponding to the plurality of deposition sites on the array surface, such that when the charge is applied to a circuit path that accesses a respective electrode pad, the charge on the respective electrode pad forms an electric field through the membrane sheet at a corresponding deposition site to create the electric field differential.

22. The apparatus of claim 21, wherein the membrane sheet is chargeable on the array surface with an electrostatic charge that is different from the charge applicable to the circuit path, such that when both charges are applied to the substrate, the array surface comprises both of the electrostatic charge and the electric field.

23. The apparatus of claim 16, further comprising control electronics that one or more of supplies, removes and varies the charge provided to the circuitry, wherein the control electronics optionally permit a controlled volume of the material to accrete on the electrically influenced deposition site or the other location on the array surface.

24. A deposition mask having an electrostatically changeable mask pattern comprising the apparatus of claim 16, wherein the changeable mask pattern comprises a selected set of deposition sites from the plurality of deposition sites for each chemical or biochemical material to be deposited and for each incidence that a same chemical or biochemical material is deposited during the chemical array fabrication, each selected set having at least one electrically influenced deposition site, the at least one electrically influenced deposition site of each selected set being a same deposition site or a different deposition site of the plurality relative to other selected sets.

25. The deposition mask of claim 24, further comprising:
control electronics connectable to the substrate circuitry, the control electronics comprising a source of charge, the control electronics independently accessing each selected set of deposition sites with a respective portion of the circuitry that corresponds to an accessed selected set of deposition sites to one or more of supply, remove or vary the charge that electrically influences the accessed selected set of deposition sites.

26. The deposition mask of claim 25, wherein the substrate further comprises a membrane sheet of an electrically nonconductive material, the membrane sheet providing the array surface, the plurality of deposition sites being on the array surface of the membrane sheet, the charge supplied to the respective portion of the circuitry provides an electric field through the membrane sheet at the selected set of deposition sites indirectly corresponding to the respective charged circuitry portion.

27. A system for electrostatically guiding the material in chemical array fabrication comprising the apparatus of claim 16, and further comprising:
a source of ionized droplets of the material; and
control electronics comprising a source of charge, the control electronics one or more of supplies, removes and varies the charge applied to the circuitry, the control electronics optionally comprising charge-sensing circuitry that monitors a volume of the material that is accreted on the array surface.

28. The system of claim 27, wherein the source of ionized droplets comprises one or more of an aerosol generator, a fogger or a sprayer that produces ionized microdroplets of the material.

29. The system of claim 27, wherein the source of ionized droplets comprises an ink-jet print head that produces ionized droplets of the material at each nozzle of the print head.

30. The system of claim 27, further comprising a cell enclosing at least the array surface of the substrate, the cell having a volume space adjacent the array surface and a port through which the ionized material droplets are introduced to the volume space.

31. The system of claim 30, wherein the port is a narrow slot, such that the cell is a Hele Shaw cell, which controls flow parameters of the introduced ionized droplets.

32. A method of electrostatically guiding a chemical or biochemical material to be deposited in chemical array fabrication comprising:
generating an electric charge or field differential on an array, the array comprising a plurality of deposition sites on a surface of the array, the plurality of sites being in a spatially addressable array pattern, the electric charge or field differential comprising a first charge that electrically influences a selected set of deposition sites of the plurality, and a second charge on an area surrounding the selected set, the first charge being different from the second charge; and
exposing the array to an ionized droplet of the chemical or biochemical material, the ionized droplet having a charge, such that the electric charge or field differential guides the ionized droplet to preferentially deposit on either the selected set of influenced deposition sites or the surrounding area due to an electrostatic force.

33. The method of claim 32, wherein generating an electric charge or field differential comprises:
providing an array substrate that comprises an electrically nonconductive photoconductive layer over an underlying electrically conductive layer;
applying the second charge to a surface of the photoconductive layer, the photoconductive layer being protected from exposure to light;
connecting the conductive layer to a first charge source or sink; and
illuminating with light unprotected regions on the surface of the photoconductive layer corresponding to the selected set of deposition sites, such that the illuminated regions are electrically conductive while being illuminated, the electrically conductive regions dissipating the second charge into the conductive layer and acquiring the first charge of the underlying conductive layer,
wherein a non-illuminated portion of the photoconductive layer is the surrounding area that has the second charge.

34. The method of claim 32, wherein generating an electric charge or field differential comprises applying the first charge to regions on the array corresponding to the deposition sites, wherein applying the first charge to regions comprises:
providing an array substrate of an electrically nonconductive material that comprises the plurality of deposition sites and further comprises electrically conductive circuit paths embedded in the substrate and electrically conductive electrode pads on or in the substrate adjacent to the array surface, each electrode pad being interconnected to a circuit path, each deposition site of the plurality having a directly or indirectly corresponding electrode pad; and
applying the first charge to the circuit paths that interconnect to a selected set of the electrode pads, the selected set of charged electrode pads corresponding to the selected set of influenced deposition sites,
wherein the surrounding area is a portion of the array surface not influenced by the first charge.

35. The method of claim 34, wherein the array substrate further comprises a membrane sheet on the array substrate, the selected set of deposition sites being on the array surface of the membrane sheet, the selected set of influenced deposition sites indirectly corresponding to the charged set of electrode pads on or in the array substrate, the charged set of electrode pads providing an electric field through the membrane sheet at locations that correspond to the selected set of influenced deposition sites.

36. The method of claim 32, wherein exposing the array to an ionized droplet of the material comprises dispensing ionized droplets of the material to the array surface using an ink-jet print head.

37. The method of claim 32, wherein exposing the array to an ionized droplet of the material comprises generating an aerosol, a fog or a spray of ionized microdroplets of the material and introducing the generated ionized microdroplets adjacent to the array surface.

38. The method of claim 32, further comprising varying the first charge that influences a deposition site of the selected set of deposition sites to control accretion of the material on the influenced deposition site.

39. The method of claim 32, further comprising repeating generating an electric charge or field differential with the first charge for a subsequent selected set of deposition sites for each chemical or biochemical material to be subsequently deposited and for each incidence that a same chemical or biochemical material is deposited during the chemical array fabrication, and repeating exposing the array to ionized droplets of each material, wherein each subsequent selected set is one or both of a same set or a different set relative to a previously selected set of deposition sites.

40. The method of claim 32, wherein after the array is fabricated, the method optionally comprises one or more of:
exposing the fabricated array to a chemical or biological sample;
reading the exposed array;
forwarding a result of the reading to a remote location; and
transmitting data representing a result of the reading.
